(19) United States
(12) Patent Application Publication
(10) Pub. No.: US 2011/0014273 A1
(43) Pub. Date: Jan. 20, 2011

(54) Method of Treating BCC

(76) Inventor: Helmut Brunar, Vienna (AT)

Correspondence Address: LOZA & LOZA LLP, 305 North Second Ave., #127, Upland, CA, 91786, US

(21) Appl. No.: 12/836,379

(22) Filed: Jul. 14, 2010

Related U.S. Application Data
(60) Provisional application No. 61/225,820, filed on Jul. 15, 2009.

(51) Int. Cl. A61K 31/7036; A61K 9/127; C07H 15/236; A61P 31/04

(52) U.S. Cl. 424/450; 514/40; 536/13.6

(57) ABSTRACT

A method of treating chronic infection with B. cepacia complex (BCC) in a patient suffering from cystic fibrosis (CF) comprising administering a fluidosomal formulation of tobramycin to the patient.

### CROSS-REFERENCE TO RELATED APPLICATIONS

This application claims the benefit of priority from U.S. Provisional patent application Ser. No. 61/225,820, filed on Jul. 15, 2009 and entitled “Method of Treating BCC,” the disclosure of which is incorporated herein by reference in its entirety.

### FIELD OF THE INVENTION

The present invention relates to a method of treating bacterial infection, and in particular, a method of treating infection resulting from highly resistant bacterial strains.

### BACKGROUND OF THE INVENTION

Cystic fibrosis (CF) is a life-threatening disorder that causes severe lung damage and nutritional deficiencies. It is an inherited autosomal recessive genetic disease occurring in all ethnic groups. CF is caused by mutations in a single gene on chromosome seven, which encodes the cystic fibrosis transmembrane conductance regulator (CFTR). This protein functions as an ion channel across the cell membrane. Such channels are found in tissues that produce mucus, sweat, saliva, tears and digestive enzymes. Chloride, a component of salt, is transported through the channels in response to cellular signals. The transport of chloride helps to control the osmotic movement of water in tissues and maintains the fluidity of mucus and other secretions.

The CFTR protein also regulates the function of other ion channels, for example sodium, across cell membranes. Normal functioning of these channels is necessary for organs such as the lungs and pancreas to function properly. CFTR mutations result in the formation of malfunctioning CFTR protein and affect epithelial ion and water transport mainly in the cells of the respiratory, gastrointestinal, hepatobiliary and reproductive tracts. The most common symptoms include very salty-tasting skin, persistent coughing, wheezing or pneumonia, excessive appetite with poor weight gain and bulky stools. Typically, symptoms appear in infancy, either as meconium ileus (a form of intestinal obstruction in newborns), or as poor weight gain at 4 to 6 weeks. More unusually, symptoms become apparent in early childhood in the form of persistent coughing, wheezing and respiratory tract infection. Respiratory failure is the most dangerous consequence of CF.

The most characteristic respiratory symptom of CF is the excessive production of thick, sticky mucus in the airways, which impedes breathing and provides an ideal breeding ground for many microorganisms. A major feature of CF lung disease is chronic endobronchial infection with Pseudomonas aeruginosa, a gram-negative bacterium that is predominantly observed in the second decade of life of patients with CF. Once pulmonary infection is established, it is difficult to eradicate the bacteria. Such infection is associated with progressive deterioration in lung function and mortality, with patients losing an average of 2% of their lung function per year.

An important class of antibiotics used to treat infection by organisms such as P. aeruginosa is the aminoglycoside class of compounds, an effective member of which is tobramycin. When treating P. aeruginosa in patients with CF, however, high doses are needed to ensure that sufficient amounts of antibiotic reach the lung because of poor penetration and decreased antimicrobial activity in purulent sputum. A breakthrough in the treatment of lung infections in CF was the administration of tobramycin as an aerosol with the aid of a nebulizer. aerosolized tobramycin allows a more direct delivery to the site of infection and can be given at higher doses than would be safe to administer systemically. In 1997, the FDA approved non-liposomal tobramycin solution for inhalation under the trade name TOBI® for the management of CF patients with P. aeruginosa. This was the first marketed aerosol form of an antibiotic for the management of CF.

Although conventional tobramycin for inhalation (TOBI®) has an established safety profile, the aminoglycoside class of antibiotics are associated with hearing loss, dizziness, kidney damage, and harm to the fetus. In addition, conventional tobramycin formulations rarely eradicate P. aeruginosa infections, even before the appearance of tobramycin-resistant mutants. Mucoid variants of P. aeruginosa are particularly resistant and are the main cause of morbidity and mortality among patients with CF. Other resistant bacterial infections have also emerged in CF patients, including organisms of the Burkholderia cepacia complex (BCC).

BCC is a group of catalase-producing, non-lactose-fermenting Gram-negative bacteria composed of at least nine different species, including B. cepacia, B. multivorans, B. cenocepacia, B. vietnamiensis, B. stabilis, B. ambifaria, B. dolosa, B. anthina, and B. pyrrocinia. B. cepacia is an important human pathogen which most often causes pneumonia in immunocompromised individuals with underlying lung disease (such as cystic fibrosis or chronic granulomatous disease). Diagnosis of BCC involves culturing the bacteria from clinical specimens such as sputum or blood. BCC organisms are naturally resistant to many common antibiotics including aminoglycosides and polymyxin B and this fact is exploited in the identification of the organism. Thus, means to identify infection with BCC include in vitro culture assays with aminoglycosides to determine the intrinsic resistance.

The prevalence of BCC has remained relatively stable among CF patients in the United States during the past several years; about 3 to 4% of patients are infected (Cystic Fibrosis Foundation Patient Registry), although prevalence is much higher in certain centers. When stratified by age, however, 1 in 10 adults with CF have had a positive culture. Comprehensive taxonomic studies confirmed that this single species was actually composed of bacteria that, although closely related and phenotypically similar, had sufficient genetic differences to warrant their division into several new species. By taxonomic convention, these new species were referred to as genomovars until distinguishing phenotypic characteristics were identified. Identification of distinct phenotypes allowed the proposal of formal binomial designations for each species. Currently, there are nine species in what is now collectively referred to as the B. cepacia complex. Although all members of the complex have been cultured from CF sputum, B. cenocepacia (genomovar III), B. multivorans (genomovar II), and B. vietnamiensis (genomovar V) account for the majority of isolates, 50%, 35%, and 5%, respectively.

Infection with BCC has been identified as an independent negative prognostic indicator in CF. Patients may even develop a cepacia syndrome.

It has been shown that aminoglycoside antibiotics, like tobramycin, were counterproductive, because it could even induce bacterial biofilm formation, which again contributes to the BCC resistance to antibiotics (Hoffman et al 436/25 August 2005/doi:10.1038/nature03912: page 1171-1175). Biofilms are adherent aggregates of bacterial cells, that can form in the airways of patients with CF, thus contributing to bacterial persistence in chronic infections.

One way to improve the safety and effectiveness of antibiotics is to encapsulate the drug in liposomes that are composed of naturally occurring or synthetic phospholipids. In this regard, EP0806941 B1 and Beaulac et al. (Journal of Antimicrobial Chemotherapy (1998) 41, 35-41) teach that liposomal tobramycin, encapsulated in a low rigidity liposomal formulation which is free of cholesterol, comprising neutral and anionic phospholipids at a molar ratio of 5:1 to 20:1 (mean T_{c} is below 37° C.) is effective in vitro to treat a single strain of B. cepacia. Subsequently, in Marier et al. (Antimicrob Agents Chemother (2002) 46: 3776-3781), growth of a single strain of B. cepacia, namely strain BC-1368, was inhibited in a rat model of chronic lung infection using liposomal tobramycin, and growth of B. cenocepacia strains was also inhibited using liposomal tobramycin comprising cholesterol (Halwani et al. , Journal of Antimicrobial Chemotherapy (2007) 60, 760-769).

However, emerging and unusual gram negative bacterial species that exhibit more severe forms of infection have recently been identified, among them the inherently resistant BCC in CF patients (Davies et al. Semin Respir Crit Care Med (2007) 28(3):312-321). People with CF have chronic airway infection and are frequently exposed to antibiotics, which often leads to the emergence of resistant organisms. Though these bacteria are not usually pathogenic for healthy persons, people with CF are highly susceptible to chronic infections with such resistant BCC, which is hardly manageable by antibiotics. In particular, BCC organisms produce biofilms in vivo, thus these organisms are highly resistant to aminoglycosides.

In view of the foregoing, there is a need to develop a method of treating such resistant bacterial strains in patients with CF and other related conditions.

### SUMMARY OF THE INVENTION

A method of treating chronic infection with B. cepacia complex (BCC) in a patient suffering from cystic fibrosis (CF) or resistant pulmonary infection is provided that comprises administering a fluidosomal formulation of tobramycin to the patient.

The preferred method according to the invention refers to BCC that is resistant to treatment with free, non-liposomal, tobramycin.

Another preferred method according to the invention refers to a patient who has developed a biofilm of BCC cells.

Another preferred method according to the invention refers to BCC having a minimal inhibitory concentration of at least 10 μg/ml.

According to a preferred embodiment the method according to the invention refers to fluidosomal tobramycin that is provided at a dose of 30-600 mg/day. The preferred fluidosomal tobramycin is a liposomal preparation having a phase transition temperature of below 37° C.

Another preferred fluidosomal tobramycin is a liposomal preparation comprising DPPC and DMPG in a ratio of 10:1 to 15:1.

According to the invention there is further provided the use of fluidosomal tobramycin in the manufacture of a medicament for treatment of BCC infection in patients suffering from cystic fibrosis.

The preferred use according to the invention refers to the patient, who is refractory to treatment with free tobramycin.

These and other aspects of the invention will become apparent in the following description.

### DETAILED DESCRIPTION OF THE INVENTION

A method of treating chronic infection with B. cepacia complex (BCC) in a patient suffering from cystic fibrosis (CF) is provided. The method comprises administration to the patient a fluid liposomal, also called fluidosomal formulation of tobramycin. The method may also be used to treat resistant bacterial strains in general.

The term “resistant bacterial strain” is meant to encompass gram positive and gram negative bacteria that are intrinsically resistant to a broad range of antimicrobial agents, as well as to nonoxidative killing by human phagocytic cells. Multi-drug resistance is minimally defined as resistance to all of the agents in two of three classes of antibiotics, such as quinolones, aminoglycosides, and b-lactam agents, including monobactams and carbapenems. Intrinsic resistance is due to mechanisms other than specific drug metabolizing pathways and denotes a general impermeability of the bacterial cell to many classes of antimicrobials. Typically, intrinsically resistant organisms are resistant to large concentrations of aminoglycoside and polymyxin antibiotics due to not well defined unusual properties of the bacterial cell envelope, including its lipopolysaccharide component. A typical minimal inhibition concentration (MIC) of resistant bacteria is at least 10 82 g/ml, in more resistant cases, at least 20, at least 30, at least 40, at least 50 or even at least 60 μg/ml, in some very resistant cases the MIC is even higher than 100 μg/ml. Resistance may be determined by appropriate in vitro culture techniques, using specific antibiotics concentrations and cultivation conditions. Another way to determine resistance is by the observation, that patients are refractory to the antibiotic treatment, thus need alternative treatment. For example, a patient refractory to treatment with free tobramycin, such as TOBI®, is deemed to have a resistant bacterial infection.

The term “patient” as used herein includes a human or other mammalian subject in need of treatment according to the present methods.

Examples of resistant bacterial strains include strains of P. aeruginosa, Burkholderia cepacia complex such as B. cenocepacia, B. dolosa, B. multivorans and B. vietnamiensis, or ET12 strains, Stenotrophomonas maltophilia, Achromobacter (Alcaligenes) xylosoxidans, Ralstonia species and Pandoraea species, and gram-positive bacteria including methicillin-resistant S. aureus (MRSA) and vancomycin-resistant enterococci (VRE) as well as penicillin and/or cephalosporin resistant Streptococci pneumoniae.

According to the present treatment method, fluidosomal tobramycin is administered to a mammal to treat bacterial infection resulting from at least one resistant bacterial strain, and potentially more than one resistant bacterial strain, e.g. two or more related or unrelated resistant bacterial strains. Thus, the method may be utilized to treat bacterial infection by a resistant P. aeruginosa strain in combination with a resistant strain of the Burkholderia cepacia complex, or infection by multiple resistant strains of the Burkholderia cepacia complex.

The term “mammal” is used herein to refer to both human and non-human mammals, including domestic and wild animals.

The fluid liposomal formulation utilized in the present method preferably is free from activating agent such as cholesterol, and comprises a combination of lipids, such as phospholipids. Preferably a neutral lipid and an anionic lipid is used to formulate tobramycin (4-O-(3-amino-3-deoxy-α-D-glucopyranosyl-2-deoxy-6-O-(2,6-diamino-2,3,6-trideoxy-α-D-ribo-hexopyranosyl)-L-streptamine). Examples of suitable neutral lipids include a phosphatidylcholine such as dipalmitoylphosphatidylcholine (DPPC). Examples of suitable anionic lipids include a phosphatidylglycerol such as dimirystoyl phosphatidylglycerol (DMPG). The ratio of neutral lipid to anionic lipid in the formulation is in the range of about 5:1 to 20:1, and more preferably in the range of about 10:1 to 15:1. The ratio of total lipid to tobramycin is in the range of about 10:1 to 1:1.

The ratio of the individual phospholipids may be selected that determine the respective gel-liquid crystalline transition temperature. The composition is preferably provided that has a phase transition temperature (Tc) of below 37° C., more preferably between room temperature or 25° C. and 35° C., as determined by differential scanning calorimetry (DSC). Thus, the preferred lipids as used according to the invention have a relatively low phase transition temperature, such that the fluidosomal tobramycin formulation as used according to the invention has the Tc in the preferred range or a low rigidity.

The fluidosomal formulation may be prepared using established methodology including rehydration or lyophilization, homogenization, extrusion under pressure, diafiltration/ultrafiltration and/or sterile filtration. According to a preferred embodiment the fluidosomal tobramycin preparation is produced by dissolving the lipids in an organic solvent, evaporating the solvent to obtain a lipid film, hydrating with an aqueous solution of tobramycin to form multilamellar liposomes, lyophilizing, rehydrating and extrusion through successively smaller pore polycarbonate membranes.

The preferred fluidosomal formulation comprises liposomes ranging in size from about 0.2 μm to 0.6 μm in combination with a pharmaceutically acceptable carrier.

Preferably multilamellar liposomes entrapping tobramycin are used according to the invention. As one of skill in the art will appreciate, the fluidosomal tobramycin formulation may be administered to a mammal in need of treatment by various routes including by injection (e.g., intravenous, intraperitoneal, intramuscular, subcutaneous, intraauricular, intramammary, intraurethrally, etc.), orally, topically (e.g., on afflicted areas), by absorption through epithelial or mucocutaneous linings (e.g., ocular epithelia, oral mucosa, rectal and vaginal epithelial linings, the respiratory tract linings, nasopharyngeal mucosa, intestinal mucosa, etc.) and by inhalation.

The pharmaceutically acceptable carrier, with which the liposomes are combined, will vary with the selected route of administration. The expression “pharmaceutically acceptable” means acceptable for use in the pharmaceutical and veterinary arts, i.e. not being unacceptably toxic or otherwise unsuitable. Examples of pharmaceutically acceptable carriers are those used conventionally in liposomal formulations, such as diluents, excipients and the like. Reference may be made to “Remington's: The Science and Practice of Pharmacy”, 21^{st} Ed., Lippincott Williams & Wilkins, 2005, for guidance on drug formulations generally. In one embodiment of the invention, the liposomes are formulated for administration by infusion, or by injection either subcutaneously or intravenously, and are accordingly prepared in an aqueous solution in sterile and pyrogen-free form and optionally buffered or made isotonic, e.g. prepared in distilled water or, more desirably, in saline, phosphate-buffered saline or 5% dextrose solution. In another embodiment, the liposomes are formulated for administration by inhalation and combined with one or more propellant adjuvants to form an aerosol.

It was surprisingly found that the fluidosomal tobramycin formulation according to the invention effectively could reduce the number of strongly resistant bacteria, such as BCC, thus, be provided as an effective antibiotic. This was particularly surprising in view of the findings that tobramycin could induce a biofilm in CF patients, thus would be counterproductive in treating chronic infections in CF patients.

The mode of action is still not entirely clear, but pre-clinical data indicate a novel mode of action: tobramycin containing liposomes seem to merge with the cell membrane of the pathogen. In this way the entire load of tobramycin contained in the fluidosomal tobramycin is released into the bacterial cell. Additionally our data indicate that bacterial rescue mechanisms to pump tobramycin out of the cell are inhibited by the fusion process. The efficient delivery and maximum release of tobramycin into the bacterial cell together with inhibition of the clearance mechanism indicates a very efficient therapeutic effect of fluidosomal tobramycin.

Fluidosomal technology uses biocompatible lipids endogenous to the lung that are formulated into small liposomes. This nanocapsule platform offers wide-ranging potential for unmet medical needs, including chronic respiratory infections of the lung. In case of fluidosomal tobramycin, the interaction between tobramycin and the microbial cell is triggered when the liposomes undergo a fusion process with the outer membrane of the bacterial cell wall. Tobramycin then penetrates into the inner cell compartment and triggers killing of the bacterial cell.

The present fluidosomal tobramycin formulation is preferably administered to a mammal in the treatment of infection by resistant bacterial strains in a therapeutically effective amount. The term “therapeutically effective amount” is an amount determined to be effective to treat the bacterial infection which is not toxic or otherwise unacceptable for use in a mammal. As one of skill in the art will appreciate, acceptable dosages for the treatment of resistant bacterial infection in a mammal may readily be determined using routine methods. In this regard, liposomal tobramycin dosages in the range of about 30-600 mg/day, preferably 40-500 mg/day, may be used to treat a mammal infected with a resistant bacterial infection.

The present fluidosomal tobramycin formulation is particularly useful for the treatment of pulmonary infection by resistant bacterial infection by administration of an inhalable formulation.

Embodiments of the invention are described by reference to the following specific example which is not to be construed as limiting.

### Example 1

### Manufacture of Fluidosomal Tobramycin

As an example, liposomal tobramycin was made as follows: Tobramycin is gradually added to water for injection (WFI) under stirring and it is stirred until a clear solution is obtained. Whilst mixing, the pH of the solution is checked and adjusted with 6M hydrochloric acid to 7.4. The salts (potassium chloride, potassium dihydrogen phosphate anhydrous, disodium hydrogen phosphate anhydrous and sodium chloride) are added to the tobramycin solution under stirring. After adding the salts, the solution is stirred until clear. Under vigorous stirring, the solution is heated to 45° C. ±5° C. and the phospholipid mixture is slowly added and pH adjusted. Following dispersion of the phospholipids, the dispersion is homogenised: it is extruded through the homogenizer for a minimum of 3.5 hrs. The homogenized dispersion is filtered through sterile 0.20 μm filters prior to filling into vials under aseptic conditions and a pressure of 1200 mbar.

Table 1 below provides the qualitative and quantitative composition per 5 mL.

TABLE 1

| | | Quantity | |
|---|---|---|---|
| Component | Function | per vial | |
| tobramycin | drug substance | 150.0 | mg |
| 1,2-dipalmitoyl-sn-glycero-3- | excipients | 850.00 | mg |
| phosphocholine (DPPC)/ | | | |
| 1,2-dimyristoyl-sn-glyco-3- | | | |
| phosphoglycerol, sodium salt | | | |
| (DMPG) 10:1 (w/w) | | | |
| potassium dihydrogen phosphate | excipient | 1.3 | mg |
| anhydrous (KH₂PO₄) | | | |
| disodium hydrogen phosphate | excipient | 8.00 | mg |
| anhydrous (Na₂HPO₄) | | | |
| 6 M hydrochloric acid (HCl)* | pH adjustor | 150.00 | mg |
| sodium chloride | excipient | 31.46 | mg |
| potassium chloride | excipient | 0.80 | mg |
| water for injection (WFI)** | diluent | 5.10 | g |

*The 6 M hydrochloric acid is prepared from 37% hydrochloric acid (Ph. Eur.) with the addition of a suitable quantity of WFI (complies with Ph. Eur Monograph for “Water for injections in bulk”)
**Complies with Ph. Eur Monograph for “Water for injections in bulk”

### Example 2

### Efficacy of Fluidosomal Tobramycin Against P. Aeruginosa Infection

Male Sprague-Dawley rats were inoculated with 10⁶ colony forming units (cfu) of a mucoid P. aeruginosa variant PA 508 (MIC: 1 mg/L) to assess differences in pharmacokinetics (Beaulac et al 1996, see above) and efficacy between fluidosomal tobramycin according to the invention and the conventional formulation of tobramycin (TOBI®' Novartis).

Conventional tobramycin for inhalation (TOBI®) or fluidosomal tobramycin was intratracheally administered in single (490 μg tobramycin) or multiple dose experiments (490 μg/day tobramycin for 4 days) to 78 and 76 infected rats, respectively. Liposomal PBS served as negative control. Following tobramycin treatments, rats were killed at different time points and pulmonary samples were taken. Entire lungs of killed rats were used to determine the residual cfu of P. aeruginosa and tobramycin amounts by HPLC in lungs. Pearson χ² analyses were carried out on cfu data distributed in the following categories: below 10³, 10³-10⁵, and above 10⁵ cfu. In the single dose experiments about 90% of the observations were above 10⁵ cfu for both formulations. Significant differences in cfu distribution were observed after multiple treatment only (P=0.037), with 9.4% of the observations falling below 10³ cfu for the conventional formulation (TOBI®) against 28.1% for fluidosomal tobramycin (Table 2).

TABLE 2
Distribution of residual cfu of P. aeruginosa in lungs following the
intratracheal administration of the conventional formulation of
tobramycin (TOBI ®) and fluidosomal tobramycin in the single and
multiple dose experiments

| | TOBI ® | | fluidosomal tobramycin | |
|---|---|---|---|---|
| | Single dose | Multiple dose | Single dose | Multiple dose |
| Residual | No. (%) | No. (%) | No. (%) | No. (%) |
| cfu | of rats | of rats | of rats | of rats |
| >10⁵ | 32 (89.0) | 12 (37.5) | 32 (89.0) | 15 (46.9)* |
| 10³-10⁵ | 2 (5.5) | 17 (53.1) | 4 (11.0) | 8 (25.0)* |
| <10³ | 2 (5.5) | 3 (9.4) | 0 (0.0) | 9 (28.1)* |

*P < 0.05 versus TOBI ®

Fluidosomal tobramycin has the net advantage of improving drug exposure at the site of infections by prolonging its pulmonary distributional and elimination half-lives. As a result of this, the fluidosomal tobramycin resulted in greater efficacy than that of TOBI® after multiple treatments. These results support the use of fluidosomal tobramycin to provide more aggressive treatments for the management of P. aeruginosa pulmonary infections in CF patients.

### Example 3

### Efficacy of Fluidosomal Tobramycin Against B. Cepacia Infection

In total 78 male Sprague-Dawley rats were inoculated intratracheally with 10⁶ cfu of a very resistant strain of B. cepacia (strain BC 1368; MIC: 128 μg/ml) to establish lung infection. Six days later a 1.200 μg dose of tobramycin was administered intratracheally as a fluidosomal or a conventional formulation. Pearson χ² analyses were performed on residual cfu data distributed in the following categories: <10³, 10³-10⁵, and >10⁵.

Differences in cfu data between formulations showed a statistical trend (P<0.10) in favour of the fluidosomal formulation when data of 75 rats from all time points were used, and statistically significant differences were found after 12 h (P<0.05), with greater eradication achieved with the liposomal formulation (Table 3). In conclusion, intratracheal administration of tobramycin in fluidosomes was associated with an apparent trend for a prolonged efficacy against B. cepacia. These results support the hypothesis that inhalation of fluidosomal tobramycin may also improve the management of chronic pulmonary infections caused by resistant B. cepacia strains in patients with CF.

TABLE 3
Distribution of residual cfu of B. cepacia in lungs following treatment with conventional
formulation of tobramycin (TOBI ®) or fluidosomal formulation of tobramycin

| | Time (h) | | | | | | | | | | |
|---|---|---|---|---|---|---|---|---|---|---|---|
| | 0-24 h | | | | 0-12 | | | | 12-14 | | |
| | Formulation | | | | | | | | | | |
| | Fluidosomal | | TOBI ® | | Fluidosomal | | TOBI ® | | Fluidosomal | TOBI ® | |
| | No. (%) | | No. (%) | | No. (%) | | No. (%) | | No. (%) | No. (%) | |
| Residual cfu | of rats | | of rats | | of rats | | of rats | | of rats | of rats | |
| >10⁵ | 14^{a} | (35.9) | 7 | (19.4) | 9 | (37.5) | 4 | (19.0) | 5^{b} (33.3) | 3 | (20.0) |
| 10³-10⁵ | 18^{a} | (46.2) | 26 | (72.2) | 12 | (50.0) | 14 | (66.6) | 6^{b} (40.0) | 12 | (80.0) |
| <10³ | 7^{a} | (17.9) | 3 | (8.3) | 3 | (12.5) | 3 | (14.3) | 4^{b} (26.7) | 0 | (0.0) |

^{a}P < 0.05 compared with TOBI ®
^{b}P < 0.10 compared with TOBI ®
Note:
CFU <10³ corresponding to >99.9% kill
CFU between 10³-10⁵ corresponding to 90.0-99.9% kill
CFU >10⁵ corresponding to <90.0% kill

### Example 4

### Evaluation of Immunogenicity of Inhaled Fluidosomal Tobramycin

Although liposomes are usually non-toxic and non-immunogenic, the local treatment of pulmonary infections with fluid liposomes, may affect their metabolism and may cause potential side effects. In addition, several factors have been proven to influence the immunogenic properties of liposomes, e.g. charge on the liposomal surface, lipid composition, size and T_{c}. Therefore, it was important to study local/mucosal and systemic immune responses following the inhalative administration.

The systemic and mucosal immunogenicity of liposomes composed of DPPC and DMPG at a ratio of 18:1 (w/w) were assessed by conducting repeated intraperitoneal (i.p.) and intratracheal (i.t.) immunizations in BALB/c mouse (Sachetelli et al., Biochimica et Biophysica Acta 1428 (1999) 334-340). Immune responses (IgM, IgG, IgA) were examined in sera and bronchoalveolar lavages (BALs) of different concentrations of liposomes containing tobramycin from 6 i.p. and 3 consecutive i.t. immunizations. Each group was tested for the presence of antibodies against liposomes and tobramycin.

I.P. immunization: Twenty-eight mice were i.p. immunized with different concentrations of liposomes (0.8-4 μmol) containing tobramycin (0.026 -0.2 mg) or PBS. Five mice were hyperimmunized with liposome-PBS containing lipid A to get positive controls. Mouse sera collected and pooled prior to the start of immunization were used as a negative control. Booster shots were administered after 7, 14, 21, 28 and 35 days or 7, 14, 21, 28, 35, 42, and 49 days. Sera were collected 7 days after the last boost.

After subtraction of ELISA values obtained with pre-immune sera, only very low levels of antibodies to liposome and tobramycin could be detected. The presence of tobramycin or PBS in the liposomes did not influence the production of antibodies against liposomes or tobramycin. This showed that encapsulation of tobramycin had no adjuvant immunogenic effect.

For i.t. immunization 0.025 mL of liposomes, free tobramycin or PBS were intratracheally instilled in anaesthetized mice via a catheter located just above the alveolar tree, followed by a bolus of air to disperse the solution into the lungs. Mice were immunized every 2 weeks for a total of 3 injections. Blood was taken 7 days after the third immunization. To determine the production of IgA antibodies in BALs, groups were randomly separated in halves at the end of the study. Sera and BALs were tested for antibodies against liposomes and tobramycin by ELISA, but no significant levels of antibodies against liposomes or tobramycin were detected by ELISA.

This study showed that fluidosomal liposomes, also called fluidosomes, are not immunogenic, even after i.p. and i.t. hyperimmunization, whether or not they were associated with tobramycin. This is clinically important as it demonstrates that the potential benefits of fluidosomes should not be compromised by associated immune responses. Overall, since no significant mucosal and serum immune responses against liposomes or tobramycin were detected, these data suggest that liposome-encapsulated tobramycin could be administered repeatedly without adverse immune response.

### Example 5

### Dose Range of Inhaled Fluidosomal Tobramycin

A 14-day dose-range finding study in rats was conducted at the Fraunhofer Institute of Toxicology and Experimental Medicine (ITEM), Hannover (Germany) to evaluate the potential toxicity of fluidosomal tobramycin and its interference with lung surfactant and pneumocytes after inhalation in rats over a period of 14 days (Fraunhofer ITEM Study No. 02N05 537, final report).

22 male and 22 female SPF-Wistar rats (Crl:WU), approximately 6 weeks of age at delivery, were purchased from Charles River Deutschland, Sulzfeld, Germany. The study consisted of 5 males and females in each treatment group. Additionally 2 males and 2 females were used as cage control for investigation of the normal histopathology of these animals.

The animals were exposed by nose only inhalation either to the vehicle or a target concentration of 300 mg/m³ tobramycin for 25, 40 or 60 minutes per day over a period of 14 days.

The actual measured total mass concentrations were 2251 mg/m³ (vehicle), 2895 mg/m³ (fluidosomal tobramycin low), 2001 mg/m³ (fluidosomal tobramycin medium), and 1955 mg/m³ (fluidosomal tobramycin high) resulting in tobramycin concentrations of 417 mg/m³, 288 mg/m³, and 282 mg/m³ in the fluidosomal tobramycin low, medium and high groups, respectively. The average tobramycin dose was 7.0, 7.7, and 11.4 mg/kg/day in the fluidosomal tobramycin low, medium and high groups, respectively. The factor between intended human dose and the animal dose was 3.3, 3.6, and 5.3 in the fluidosomal tobramycin low, medium and high groups, respectively.

No mortality occurred during the course of the study. Compared to the control (vehicle) group the lung weight was significantly higher in the female fluidosomal tobramycin treated groups.

No histopathological changes were observed in the male vehicle control and in the female untreated control group. All of the observed findings in the other groups showed a rather sporadic distribution and affected between ⅕ and ⅗ animals per group. These findings included (multi)focal alveolar histiocytosis, (multi)focal interstitial mononuclear cell infiltration, (multi)focal alveolar inflammatory cell infiltration, alveolar haemorrhage, congestion (hyperaemia) and osseous metaplasia. With the exception of congestion (score: slight to moderate), all other findings were scored as very slight (minimal). There was no statistically significant difference for any finding between the control groups and the fluidosomal tobramycin treated groups and only for congestion (hyperaemia) a slight dose-response relationship could be observed in treated males. However, all of the observed changes are typically seen also in untreated control rats of this strain and age.

In the fluidosomal tobramycin treated rats there seemed to be a slight diffuse increase of alveolar macrophages per alveolus but without formation of macrophage aggregates or signs of macrophage degeneration (=alveolar histiocytosis). This finding was considered to represent a normal physiological response to the treatment and therefore not recorded. Furthermore, pulmonary congestion (hyperaemia) may also be considered as a physiological response rather than a pathological change.

In summary, no significant inflammatory or other lesions could be observed in the fluidosomal tobramycin treated groups under the present experimental conditions.

### Example 6

### Toxicity Study of Inhaled Fluidosomal Tobramycin

The objective of this 28-day study (Fraunhofer ITEM Study No. 02G06004) was to evaluate the potential toxicity of fluidosomal tobramycin after inhalation in rats over a period of 28 days. A 28-day recovery period was included in the study design.

Based on the safety result of the 14-day dose range finding inhalation study (Fraunhofer ITEM No. 02N05 537), the dose of 11.7 mg/kg/day tobramycin, which would allow a safety factor of 5.4 to the intended human use, was chosen as the high dose for this study and the half dose was selected as the low dose.

72 male and 72 female Wistar (WU) rats were randomized into 6 groups (including 2 recovery groups) consisting of 10 or 13 rats per sex in each group. Animals were treated daily by nose only inhalation with either vehicle (empty fluidosomal liposomes), TOBI®, low or high dose fluidosomal tobramycin for 28 days.

The dosing schedule is presented in Table 4 below.

TABLE 4
Dosing schedule

| | Animals/Sex for | Animals/Sex for | Approx. Duration of |
|---|---|---|---|
| Group | Toxicity Evaluation | Kinetics | Inhalation min/day |
| 1 Control | 10 m | 3 m | 120 |
| (vehicle) | 10 f | 3 f | |
| 2 TOBI ® | 10 m | 3 m | 120 |
| | 10 f | 3 f | |
| 3 fluidosomal tobramycin low | 10 m | 3 m | 60 |
| | 10 f | 3 f | |
| 4 fluidosomal tobramycin high | 10 m | 3 m | 120 |
| | 10 f | 3 f | |
| 5 Control (vehicle) Recovery* | 10 m | | 120 |
| | 10 f | | |
| 6 fluidosomal tobramycin high | 10 m | | 120 |
| Recovery* | 10 f | | |

| | Target Concentration | Target Dose | Target Human | Factor Animal |
|---|---|---|---|---|
| | Tobramycin in the Exposure | Tobramycin | Dose Tobramycin | Dose/ |
| Group | Atmosphere mg/m³ | mg/kg/day** | mg/kg/day*** | Human Dose |
| 1 Control | — | — | — | — |
| (vehicle) | | | | |
| 2 TOBI ® | 150 | 11.7 | — | — |
| 3 fluidosomal | 150 | 5.9 | 2.15 | 2.7 |
| tobramycin low | | | | |
| 4 fluidosomal | 150 | 11.7 | 2.15 | 5.4 |
| tobramycin | | | | |
| high | | | | |
| 5 Control | — | — | — | — |
| (vehicle) | | | | |
| Recovery* | | | | |
| 6 fluidosomal | 150 | 11.7 | 2.15 | 5.4 |
| tobramycin | | | | |
| high Recovery* | | | | |

28 day recovery period
**Assumption: 100% deposition of the measured tobramycin concentration (mean body of 250 g and a minute volume calculated)
***Assumption: 100% deposition and 50% delivery of the offered tobramycin amount and mean body weight of 70 kg

All animals were observed daily for clinical signs; food and water consumption and individual body weights were monitored. Ophthalmologic evaluations (indirect ophthalmoscopy with a slit lamp) were done for all groups prior to exposure and at study end whereas recovery groups were examined only if there were findings at the end of exposure. Haematological and clinical chemistry analyses were conducted prior to inhalation and before final sacrifice. Urine analyses were performed only before final sacrifice. Blood samples were taken for toxic kinetics.

Animals were anesthetized, exsanguinated and necropsied at the day of final sacrifice.

Besides terminal body weight selected organs were weighed. Additionally, the organs and tissues of all animals were used for histopathological investigation.

The daily average exposure time was 50 minutes for the fluidosomal tobramycin low group and 100 minutes for the other groups. These chamber concentrations led to the following calculated average dose of tobramycin: TOBI®: 16.2 mg/kg/d, fluidosomal tobramycin low: 5.1 mg/kg/d, and fluidosomal tobramycin high: 11.4 mg/kg/d.

No adverse compound-related clinical signs or effects on body weight, food, and water consumption were observed in rats during the course of the study. No mortality occurred during the course of the study.

The only test and reference item induced effect in organ weights was a significant increase in absolute and relative lung weight in TOBI® and fluidosomal tobramycin high dose males only.

Relevant treatment-related changes in clinical chemistry and haematology parameters were not observed.

Statistically significant (Fisher test) histopathological findings, which could be related to the test substances, were mucosal mononuclear cell infiltration in the nasal cavity and ‘epithelial alteration’ in the larynx.

Mononuclear cell infiltration of the nasal mucosa was slightly increased in the TOBI® and high-dose fluidosomal tobramycin treatment groups as compared to the vehicle control and fluidosomal tobramycin low dose groups. It has to be emphasized that this mild inflammatory response did not result in erosions, ulcerations, degenerative or metaplastic changes of the epithelium. Although incidence and severity of the cellular infiltrates decreased, a complete reversibility was not observed at the end of the recovery period.

Other findings in the nasal cavity such as mucous (goblet) cell hyperplasia, basal cell hyperplasia or epithelial basophilic inclusions occurred at lower incidences or were incidental. These changes might have been test substance-related but could have been also spontaneous in origin.

Minimal ‘epithelial alteration’ of the larynx was exclusively observed in the TOBI® and fluidosomal tobramycin treatment groups and therefore considered to be test substance-related. This lesion completely reversed during the recovery period.

This lesion is regarded as rat specific and was frequently seen at the base of the epiglottis and is interpreted as a lesion most likely preceding laryngeal squamous metaplasia. Epithelial alteration may also occur spontaneously and is considered an ‘adaptive’ change and non-adverse because it is a minimal lesion not expected to be associated with any dysfunction of the larynx.

The incidence of mononuclear or inflammatory cell infiltration in the larynx as well as in the trachea was distributed equally over vehicle control and treatment groups. There was no reversibility of this change, which therefore is considered to be rather spontaneous in origin than related to treatment of the animals.

The observed pulmonary lesions are considered to be unrelated to the test substances used. Most lesions developed as a reaction to (multi)-focal alveolar haemorrhage. Alveolar haemorrhage itself showed a predominance in males and decreased in incidence and severity (together with the related reactive lesions) during the recovery period. The relatively high incidence of alveolar haemorrhage in the main study, is therefore considered to be related to the experimental setting (nose-only inhalation) of the study, although spontaneous alveolar haemorrhages are also not uncommonly seen in rats of this strain and age.

Taking together, the no observed adverse effect level (NOAEL) in this study was the low dose of fluidosomal tobramycin (5.1 mg/kg/day tobramycin) based on the mononuclear cell infiltration in the nasal mucosa of the fluidosomal tobramycin high dose group, the only relevant test item exposure related finding. Furthermore, the study showed that the exposure to TOBI® and fluidosomal tobramycin high dose produced similar results in terms of toxicity.

### Example 7

### Human Dose Studies in Healthy Volunteers

A phase I single dose study with fluidosomal tobramycin for inhaled administration to healthy volunteers was conducted. The trial was designed as an open, randomized, three-way cross over study to evaluate safety, tolerability and pharmacokinetics of the liposomal formulation fluidosomal tobramycin in comparison to the marketed formulation of conventional tobramycin for inhalation (TOBI®).

Overall 32 subjects were enrolled in this study. The first part of the study started in November 2005, and six healthy male volunteers received three single doses of 150 mg conventional tobramycin for inhalation (TOBI®) and 300 mg TOBI® and 150 mg fluidosomal tobramycin. 5 mL of the solutions were administered with a PARI LC PLUS nebulizer for 15 min and pharmacokinetics and pulmonary function were assessed. No safety concerns emerged after the first administration of the different tobramycin formulations. Pulmonary function parameters for example, FEV₁, were not altered in any of the different treatment groups.

26 subjects were included in part 2 of the study. Two subjects were withdrawn after having completed the first dosing session, where they received 300 mg TOBI® or 150 mg liposomal tobramycin. For both subjects the reason for withdrawal was that the investigator was not sure whether the inhalation was reliable. Both subjects were replaced, thus 24 subjects completed the study as planned. For 6 of these 24 subjects the inhalation time for liposomal tobramycin was increased to 30 minutes.

No significant safety issues were observed and fluidosomal tobramycin was generally well tolerated.

Pharmacokinetic results:

Mean plasma concentrations of tobramycin could be calculated up to 12 hours after dosing for all 4 treatments. The shape of the plasma concentration time curves showed no substantial differences between the treatments. After administration of tobramycin over 15 minutes mean concentrations increased up to 1.25 hours after the start of dosing, remained constant for the next 0.75 hours, and decreased thereafter.

After administration of liposomal tobramycin over 30 minutes mean concentrations increased up to 1.5 hours after the start of dosing, remained constant until 3.25 hours after dosing, and decreased thereafter.

All individual pre-dose plasma concentrations were below the LLQ, except for one subject, who had a pre-dose plasma concentration of 31.9 ng/mL before treatment with liposomal tobramycin inhaled for 30 min (treatment session 1), but who showed a concentration below LLQ at time point 0.25 h. Thus, it seems that these 2 samples were exchanged, but this could not be verified.

The subjects who were both withdrawn because the investigator was not sure whether the inhalation was reliable, showed different results: for one subject all plasma (and urine) concentrations were below the LLQ, thus confirming the suspicion of the investigator. Plasma concentrations were observed for the other subject however, which were comparable to those of the other subjects in the treatment group.

Overall geometric mean AUC (AUCO-inf and AUCO-t) and Cₘₐₓ were about twice as high after 300 mg TOBI® compared to both 150 mg tobramycin and 150 mg fluidosomal tobramycin inhaled for 15 minutes. Inhalation of 150 mg fluidosomal tobramycin over 30 minutes increased the AUC and Cₘₐₓ by a factor of 1.5, but the mean values were lower than after 300 mg TOBI®. Similar AUC and Cₘₐₓ values were obtained after 150 mg formulation inhaled for 15 minutes. The between-subject variability was high. The terminal half-life was comparable for all 4 treatments (mean of 3.43 to 3.73 hours). Tₘₐₓ occurred typically between 1.25 to 3.0 hours after the start of the 15 minutes inhalation (for all 3 treatments); after inhalation over 30 minutes, Tₘₐₓ varied between 1.50 and 4.25 hours after the start of dosing.

Urine parameters:

Even after a 7 day wash-out period, tobramycin was still detectable in the urine (after 150 and 300 mg). Pre-dose urinary concentrations were generally low, the observed maximum was 831 ng/mL (Subject 14, 7 days after 300 mg TOBI®).

The urinary excretion of tobramycin after treatment with 150 mg tobramycin and 150 mg fluidosomal tobramycin inhaled for 15 minutes was similar (total excretion of 13429 and 12704 μg tobramycin, respectively, within the 24 hour collection period); the excretion was slightly lower after inhalation of 150 mg fluidosomal tobramycin for 30 min (11163 μg, but the small number of subjects has to be considered). After 300 mg TOBI® the excretion was more than twice as high (33755 μg), but the variation of the urine volume as well as the urine concentration of tobramycin was very high (e.g. arithmetic mean tobramycin concentration in the urine for the collection interval 0-4 h was 23309.3 ng/mL with a standard deviation of 20345.5 ng/mL). Therefore this finding should be considered clinically irrelevant.

Pharmacokinetic conclusions:

There was no statistically significant difference in the pharmacokinetics of 150 mg tobramycin and 150 mg fluidosomal tobramycin inhaled for 15 minutes. After 300 mg TOBI® AUC and Cₘₐₓ were about twice as high than after 150 mg. Prolongation of the inhalation period of 150 mg liposomal tobramycin to 30 minutes led to an increase in AUC and Cₘₐₓ by a factor of 1.5.

The terminal half-life of tobramycin was comparable for all 4 treatments (mean of 3.43 to 3.73 hours). Tₘₐₓ occurred mainly between 1.25 and 3.0 hour after dosing. The urine excretion of tobramycin after treatment with 150 mg tobramycin and 150 mg liposomal tobramycin inhaled for 15 minutes was similar.

### Example 8

### Evaluation of the Effect of Several Liposomal Tobramycin Preparations Against Selected Strains of Bacteria

A standard time-kill kinetic method is used to evaluate the effect of several liposomal tobramycin preparations against selected strains of bacteria. Free tobramycin (Sigma), batch F461-01-001p065, batch 2, and batch 7 are tested at 0.25-, 1-, and 4-fold the MIC value for free tobramycin against 2 strains of P. aeruginosa and at concentrations of 128 and 512 μg/mL for a single isolate of B. cepacia.

Time-Kill Protocol:

From a previous susceptibility testing experiment, the liposomal test batches and control tobramycin MIC values are shown in Table 5 below. The time-kill test is conducted using the test agents shown in bold print in Table 5:

1. F461-01-001p065

2. Free tobramycin from Sigma

3. Batch 2:R/AXP-230410N

4. Batch 7:R/IV/Tobra/290410/1

TABLE 5

| | MIC (μg/mL) at 35° C. | | |
|---|---|---|---|
| Liposomal Tobramycin Batches and | B. cepacia | P. aeruginosa | P. aeruginosa |
| Test Agents | ATCC 25608 | MMX 1609 | ATCC 27853 |
| F461-01-001p065 | >64 | 0.5 | 0.25 |
| Free tobramycin (Sigma) | >64 | 0.5 | 0.5 |
| Batch 2: R/AXP-230410N | — | — | >1 |
| Batch 1: R/AXP-190410 | — | — | 0.5 |
| Batch 2: R/AXP-230410N | — | — | >1 |
| Batch 3: R/AXP-260410B | — | — | 4 |
| Batch 5: MEPACT-GR254 | — | — | 0.5 |
| Batch 6: MEPACT-GR257 | — | — | 0.5 |
| Batch 7: R/IV/Tobra/290410/1 | — | — | 8 |
| Batch 8: R/IV/Tobra/050510/1 | — | — | 8 |
| Batch 9: R/IV/Tobra/070510/E (no drug) | — | — | >8 |
| CLSI QC range (tobramycin at 35° C.) | (0.12-1) | (0.25-1) | 0.25-1 |

For P. aeruginosa time-kill testing, the compounds are tested at 0.25-, 1-, and 4-fold the MIC value for P. aeruginosa and free tobramycin (Sigma) as shown in the table above, taking into account the free tobramycin MIC value for each individual organism. For B. cepacia, the concentrations of tobramycin are 128 and 512 μg/mL. The experiment also includes an untreated growth control vessel. All drug solutions (especially the liposomal preparations) are brought to room temperature prior to use.

TABLE 6
The test organisms

| Organism | Type | ATCC No. |
|---|---|---|
| Bulkholderia cepacia | Reference strain, | 25608 |
| | TOBI ® | |
| Pseudomonas aeruginosa | Clinical | |
| Pseudomonas aeruginosa | QC strain | 27853 |

The organisms are grown overnight (approx. 18-20 h) at 35° C. on Trypticase Soy Agar and this culture is used to inoculate 10 mL of MHBII. These cultures are grown at 37° C. for 2 hr in order to achieve early exponential phase, and then adjusted (using MHB II) to equal the turbidity of a 0.5 McFarland Standard (approx. 108 CFU/mL). This adjusted cell suspension constitutes the inoculum for the assay. Tests vessels (25 mL Erlenmeyer flasks) are set up to contain 8.0 mL of MHB II, 1.0 mL of drug solution, and 1.0 mL inoculum. The target inoculum concentration is approx. 107 CFU/mL, and the time-kill study is conducted at 37° C.

Viable counts are determined at time 0, 1, 3, 6, and 24 h.

The test vessels are incubated at 37° C. on a rotary shaker (200 rpm).

A 0.5 mL sample is taken at each time point and five serial 10-fold dilutions are prepared in broth. A 50 μL sample of each dilution is applied to duplicate agar plates (form UD to 10-5). Following incubation for 20-24 h at 35° C., the colonies are counted and the number of CFU/mL calculated.

TABLE 7
The test scheme indicating the time points and test preparations
to be evaluated for two strains of P. aeruginosa.

| | Viable Count at Time | | | | |
|---|---|---|---|---|---|
| | Point (Hour) P. aeruginosa | | | | |
| Drug | 0 | 1 | 3 | 6 | 24 |
| Sigma tobramycin at 0.25x MIC | ✓ | ✓ | ✓ | ✓ | ✓ |
| Sigma tobramycin at 1x MIC | ✓ | ✓ | ✓ | ✓ | ✓ |
| Sigma tobramycin at 4x MIC | ✓ | ✓ | ✓ | ✓ | ✓ |
| Batch F461-01-001p065 at 0.25x MIC | ✓ | ✓ | ✓ | ✓ | ✓ |
| Batch F461-01-001p065 at 1x MIC | ✓ | ✓ | ✓ | ✓ | ✓ |
| Batch F461-01-001p065 at 4x MIC | ✓ | ✓ | ✓ | ✓ | ✓ |
| Batch 2 at 0.25x MIC | ✓ | ✓ | ✓ | ✓ | ✓ |
| Batch 2 at 1x MIC | ✓ | ✓ | ✓ | ✓ | ✓ |
| Batch 2 at 4x MIC | ✓ | ✓ | ✓ | ✓ | ✓ |
| Batch 7 at 0.25x MIC | ✓ | ✓ | ✓ | ✓ | ✓ |
| Batch 7 at 1x MIC | ✓ | ✓ | ✓ | ✓ | ✓ |
| Batch 7 at 4x MIC | ✓ | ✓ | ✓ | ✓ | ✓ |
| No drug growth control | ✓ | ✓ | ✓ | ✓ | ✓ |

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A method of treating chronic infection with B. cepacia complex (BCC) in a patient suffering from cystic fibrosis (CF), comprising administering a fluidosomal formulation of tobramycin to the patient.

2. The method according to claim 1, wherein said BCC is resistant to free tobramycin.

3. The method according to claim 1, wherein said patient has developed a biofilm of BCC cells.

4. The method according to claim 3, wherein said BCC has a minimal inhibitory concentration (MIC) of at least 10 μg/mL.

5. The method according to claim 1, wherein said fluidosomal tobramycin is provided to the patient at a dose of 30-600 mg/day.

6. The method according to claim 1, wherein said fluidosomal tobramycin is a liposomal preparation having a phase transition temperature of below 37° C.

7. The method according to claim 1, wherein said fluidosomal tobramycin is a liposomal preparation comprising DPPC and DMPG in a ratio of 10:1 to 15:1.

8. Use of fluidosomal tobramycin to treat BCC infection in patients suffering from cystic fibrosis.

9. The use according to claim 8, wherein the patient is refractory to treatment with free tobramycin.
